(19) United States
(12) Patent Application Publication
(10) Pub. No.: US 2011/0207631 A1
(43) Pub. Date: Aug. 25, 2011

(54) METHOD FOR PRODUCTION OF cDNA LIBRARY HAVING REDUCED CONTENT OF cDNA CLONE DERIVED FROM HIGHLY EXPRESSED GENE

(76) Inventors: Kuniyo Ohtoko, Hitachinaka (JP); Masahide Sugiyama, Kawaguchi (JP); Seishi Kato, Sagamihara (JP)

(21) Appl. No.: 13/059,927

(22) PCT Filed: Aug. 25, 2009
(86) PCT No.: PCT/JP2009/064755

§ 371 Date: Feb. 18, 2011

(30) Foreign Application Priority Data
Aug. 26, 2008 (JP) 2008-217245

(51) Int. Cl. C40B 50/06; C12N 9/12; C07H 21/00

(52) U.S. Cl. 506/26; 435/194; 536/24.3

(57) ABSTRACT

A method for efficiently constructing a cDNA library having a reduced content of cDNA clones derived from a highly expressed gene is provided. According to the method for constructing a cDNA library using a double-stranded DNA primer having oligo dT and mRNA as a template, the proportion of cDNA clones derived from a highly expressed gene in a cDNA library was decreased through coexistence with a probe having a property of binding to the mRNA of the highly expressed target gene so as to inhibit a cDNA extension reaction resulting from reverse transcriptase.

### TECHNICAL FIELD

The present invention relates to a method for constructing a cDNA library having a reduced content of cDNA clones derived from a gene that is expressed at high frequency.

### BACKGROUND ART

The full-length sequences of genomic DNAs of various organisms such as humans, mice, rice, nematodes, and yeast have been almost completely determined by genome projects. Now in the post-genome era, research is conducted to examine the kinetics of gene groups in order to macroscopically understand the role of each gene in biological phenomena. Thus, technology for comprehensive analysis of all genes is required.

It is considered that the cells composing a human body have a genome in which about 22,000 types of gene are encoded and several tens of thousands of types of genes are expressed depending on cell type (see International Human Genome Sequencing Consortium, 2004, Nature 432, 931-945). Comprehensive examination of the expression of such genes becomes increasingly important not only for elucidation of biological phenomena, but also for developing pharmaceutical products based on gene information. In particular, comprehensive and detailed analysis of information concerning the protein primary structures encoded by genes requires that DNAs complementary to mRNAs, that is, cDNAs (complementary DNAs), be obtained.

Genes are roughly classified into 3 classes based on expression levels: a group of highly expressed genes expressed at 12,000 or more copies per cell, a group of moderately expressed genes expressed at about 300 copies per cell, and a group of lowly expressed genes expressed at only about 15 copies per cell (see David J. Bertioli., et al., Nucleic Acids Research, 1995, Vol. 23, No. 21, 4520-4523). When a cDNA library is constructed using a mixture of mRNAs with different expression levels as a template, the content of the lowly expressed gene clones contained in the cDNA library is lower than that of the highly expressed gene by three or more orders of magnitude. Accordingly, comprehensive analysis of cDNA clones contained in a cDNA library by analyzing partial nucleotide sequences requires unnecessary procedures such as redundant sequencing of clones derived from the highly expressed genes. Many intracellularly expressed genes are expressed at low levels. Hence, methods for reducing the proportion of clones derived from a highly expressed gene have been developed.

One such method uses self-association of the cDNAs derived from highly expressed genes. First, a double-stranded cDNA is prepared from mRNA and then denatured to single strands. Subsequently, an annealing reaction is performed and then single-stranded cDNAs are caused to adsorb hydroxyapatite. They are then collected from a mixture of double-stranded cDNA obtained by re-association and the cDNAs that have remained as single strands. This procedure is repeated several times, and single-stranded cDNAs are collected and then cloned. Specifically, many single-stranded cDNAs are present in the case of highly expressed genes, so that they highly likely form double stranded cDNAs via annealing reaction. However, the content of single-stranded cDNAs among lowly expressed genes is low and the association frequency is low, and thus these single-stranded cDNAs remain as single strands. This method involves collecting single-stranded cDNAs derived from lowly expressed genes that have remained unassociated (see JP Patent Publication (Kokai) No. 4-108385 A (1992)).

Another method is based on hybridization with mRNA. Specifically, mRNA is transcribed from the cDNA of an existing highly expressed gene, biotinylated, and then caused to hybridize to a subject single-stranded cDNA library. cDNA not binding to the magnetic particles is recovered, and cDNA that has reacted with avidin-immobilized magnetic particles so as to bind to the biotinylated mRNA is excluded. Then, the unbound cDNA is cloned and thus a clone of the lowly expressed gene is obtained (see JP Patent Publication (Kokai) No. 2000-325080 A).

A cDNA library having an increased content of lowly expressed genes can be constructed by these methods. However, these methods require an additional step for removing the cDNA of a highly expressed gene after construction of a cDNA library from mRNA, resulting in an increased number of steps compared with those in a general cDNA library construction method. Also, the cDNA of a lowly expressed gene is nonspecifically lost, causing the problem of a reduced yield thereof.

In contrast, a method is proposed by which synthesis of 1st strand cDNA of a highly expressed gene is suppressed in a step of synthesizing 1st strand cDNA using mRNA as a template. Specifically, in the step of synthesizing 1st strand cDNA from mRNA using oligo dT primers and reverse transcriptase, a probe specifically binding to a position near the 3′ end of the mRNA of a highly expressed gene is caused to also coexist to stop 1st strand cDNA synthesis and then a probe-bound portion of the mRNA is degraded by RNaseH treatment (see JP Patent Publication (Kokai) No. 2000-37193 A). However, reverse transcriptase has activity of eliminating a probe that has bound upon transcription. Hence, it is thought that when a probe binding to an arbitrarily site of mRNA is used, 1st strand cDNA synthesis cannot be inhibited. In the above document, a probe binding to a position near the 3′ end of mRNA is used.

The use of any such method requires a step of annealing single-stranded cDNAs to form double-stranded cDNA and then integrating the resultant into an appropriate vector. Loss of the cDNA clone of a lowly expressed gene occurs during this process. Hence, it has been difficult to efficiently obtain full-length cDNA clones of a lowly expressed gene. Therefore, a method for efficiently constructing a cDNA library having a low content of a highly expressed gene with fewer steps has been desired.

### DISCLOSURE OF THE INVENTION

### Object to be Achieved by the Invention

An object of the present invention is to provide a method for efficiently constructing a cDNA library having a reduced content of cDNA derived from a single or a plurality of highly expressed genes in mRNA derived from cells or the like.

### Means for Achieving the Object

The present inventors have discovered that, when cDNA synthesis is performed using a double-stranded DNA primer as described in International Patent Publication WO2004/087916 (Pamphlet), the content of cDNA clones of a target gene can be lowered by causing a probe (for a highly expressed target gene in a nucleic acid sample) to coexist upon an extension reaction of 1st strand cDNA, so as to inhibit the extension reaction of 1st strand cDNA and to inhibit a ring-closing reaction of the conjugate of an mRNA/cDNA heteroduplex derived from the gene and a double-stranded DNA primer. Thus, the present inventors have completed the present invention. Specifically, the present invention provides a step for constructing a cDNA library having a reduced content of cDNA derived from the mRNA of a target gene that is a highly expressed gene.

The present invention relates to a method for constructing a cDNA library having a reduced content of cDNA clones derived from a target gene, for example, comprising the steps of:

(i) annealing a double-stranded DNA primer to an RNA mixture containing mRNA having a cap structure at the 5′ end and then further annealing at least one probe that binds to mRNA of the target gene so as to inhibit a reaction with reverse transcriptase;
(ii) synthesizing 1st strand cDNA from the double-stranded DNA primer using reverse transcriptase and thus preparing a conjugate of a mRNA/cDNA heteroduplex and the double-stranded DNA primer; and
(iii) ligating the 3′ end to the 5′ end of the DNA strand containing the cDNA of the conjugate of the mRNA/cDNA heteroduplex and the double-stranded DNA primer using ligase for circularization.
A highly expressed gene can be used as a target gene and a target gene can be selected based on a gene database.

The mRNA having the cap structure in step (i) is contained in a cell extract, for example. The primer sequence of a double-stranded DNA primer to be used herein contains a sequence complementary to the poly (A) sequence of mRNA having a cap structure, for example. Also, as a ligase, T4RNA ligase can be used, for example.

The above method may further comprise, between step (ii) and step (iii), step (ii′) of cleaving the conjugate of the mRNA/cDNA heteroduplex and the double-stranded DNA primer with a restriction enzyme, so as to generate a 5′ protruding end or blunt end of the double-stranded DNA primer.

Also, the above method may further comprise, following step (iii), step (iv) of substituting the RNA strand of the conjugate of the mRNA/cDNA heteroduplex and the double-stranded DNA primer with a DNA strand.

Also, in the above method, the double-stranded DNA primer may contain a replication origin, or a replication origin and a cDNA expression promoter. The probe that binds to the mRNA of a target gene so as to inhibit a reaction with reverse transcriptase is, for example, an oligonucleotide containing non-natural nucleic acids with a melting temperature (Tm value) higher by 2° C. or more than that of a complementary sequence of a partial sequence of the target gene, in which 2 or more consecutive nucleotides from the 5′ end side are composed of non-natural nucleic acids. As non-natural nucleic acids, locked nucleic acids, polyamide nucleic acids, or bridged nucleic acids (BNAs) can be used. Probes that bind to the mRNAs of target genes so as to inhibit a reaction with reverse transcriptase may target the mRNAs of different types of target gene.

Furthermore, the present invention provides, for example, a probe having a sequence complementary to an mRNA sequence. Specifically, the probe comprises an oligonucleotide that is designed so that the 3′ end has a structure modified so that it does not serve as the initiation point for an extension reaction with reverse transcriptase, 2 or more consecutive nucleotides from the 5′ end are non-natural nucleic acids, and the Tm value of the nucleotide sequence of the entire probe is higher by 2° C. or more than that of a nucleotide sequence composed of only natural nucleic acids.

An example of the probe is used for constructing a cDNA library having a reduced content of cDNA clones derived from a target gene and binds to the mRNA of the target gene so as to inhibit a reaction with reverse transcriptase.

Furthermore, the present invention provides, for example, a reagent kit for producing a cDNA library containing the above probe, a double-stranded DNA primer, reverse transcriptase, and T4 RNA ligase.

In addition, in the present invention, the term “double-stranded DNA primer” refers to a primer in which the 3′ end of one DNA strand of the double-stranded DNA protrudes and the nucleotide sequence (primer sequence) of the protruding portion has a nucleotide sequence complementary to the template mRNA sequence. This protruding portion hybridizes to template mRNA and functions as a primer when 1st strand cDNA is synthesized with reverse transcriptase. For cDNA synthesis, a double-stranded DNA primer wherein the sequence of such a protruding portion comprises 30-70 oligo dTs is used.

### Effects of the Invention

According to the present invention, a cDNA library having a reduced content of cDNA derived from the mRNA of a target gene can be provided using fewer steps than conventional methods. When a highly expressed gene is used as a target gene, the content of a lowly expressed gene becomes relatively high among the prepared cDNA clones. Thus, the complete gene information of lowly expressed genes can be efficiently obtained. Moreover, the present invention enables the cloning of the cDNA of a gene that is expressed in trace amounts and thus is cloned with difficulty by a conventional method. These genes that are expressed at low expression levels can be used as target genes for gene diagnosis for diseases or development of pharmaceutical products.

### BRIEF DESCRIPTION OF THE DRAWINGS

FIG. 1 shows basic procedures of the method for cDNA synthesis using a method for inhibiting the extension of 1st strand cDNA according to the present invention.

FIG. 2 shows the contents of genes existing in cDNA libraries constructed from RNA samples used in the examples.

FIG. 3 shows the sequences of probes used for examination.

FIG. 4 is a table showing the contents of Albumin 1 and Major urinary protein 2 in each library, as determined by hybridization.

FIG. 5 is a table showing the contents of Albumin 1 and Major urinary protein 2 in each library, as determined by sequencing of the 5′-terminal nucleotide sequence.

### BEST MODE OF CARRYING OUT THE INVENTION

This description includes part or all of the contents as disclosed in the description and/or drawings of Japanese Patent Application No. 2008-217245, which is a priority document of the present application.

Regarding the cDNA library having a reduced content of target gene-derived cDNA clones of the present invention, the cDNA library is constructed by a cDNA synthesis method using a double-stranded DNA primer as described in International Patent Publication WO2004/087916 (Pamphlet) resulting in a reduced content of a specific target gene-derived cDNA. The cDNA synthesis method is a method for synthesizing cDNA having a continuous sequence from a nucleotide adjacent to the cap structure of mRNA, for which total RNA of several micrograms can be used as a starting material. Also, cDNA can be synthesized with fewer steps without using PCR. The cDNA synthesis method is further characterized in that a full-length cDNA having the nucleotide sequence corresponding to the full-length sequence ranging from the 5′-terminal cap structure addition site to the 3′ terminal poly (A) tail of a complete mRNA guaranteed to have a continuous sequence from the nucleotide at the transcription initiation point can be synthesized with a high yield of 90% or more.

As mRNA samples, total RNA isolated from eukaryotic cells can be used. Also, mRNA having a polyA structure, which has been purified from the total RNA using an oligo dT-bound carrier may be used. Samples containing these mRNAs desirably merely undergo degradation. Specifically, such RNA sample desirably contains complete mRNA having a cap structure at the 5′ end and polyA at the 3′ end. The term “RNA mixture containing mRNA having a cap structure” in the present invention may refer to an RNA mixture substantially comprising only mRNA having a cap structure and the RNA mixture may contain mRNA and the like lacking a cap structure in addition to mRNA having a cap structure, for example.

Any gene can be selected as a target gene for reduction of the content of cDNA clones thereof in a cDNA library. In view of construction of cDNA libraries containing many lowly expressed genes, which has been performed with difficulty using conventional methods, highly expressed genes are preferred. Here, the term “highly expressed gene” refers to a gene the mRNA of which is contained at a high level in a mRNA sample because of its high expression level. The term “highly expressed gene” also refers to a gene that exists at about 12,000 copies or more per cell, for example. A gene that exists at about 300 copies or more per cell is also included herein. Many highly expressed genes are known as namely housekeeping genes or householding genes. Housekeeping genes are constantly expressed in all cells and are mainly composed of genes encoding proteins required for the survival of cells (e.g., structural proteins, enzymes of energy metabolic systems, and genes involved in mechanisms for protein synthesis, DNA replication, cell division, and the like).

Specifically, genes of such as glyceraldehyde-3-phosphate dehydrogenase (GAPDH), β-actin, a ribosomal protein, histone, phospholipase, a transferrin receptor, HPRT1, and RNA synthetase are known. These housekeeping genes can be selected as target genes. When a specific cell is used, the number or the type of a highly expressed gene differs depending on cells since a specific gene is specifically expressed at a high level in the relevant cell. Examples of such a gene that is specifically expressed at a high level in such a specific cell include albumin in hepatic cells and genes of major urinary proteins. These highly expressed genes can be selected with the use of public databases of existing genes. For example, through the use of the Bodymap (http://bodymap.ims,u-tokyo.ac.jp) database for genes expressed in each human or mouse tissue, information of highly expressed genes can be obtained for every tissue. Also, if no information exists in the existing databases, a gene expression level in an mRNA sample can be found by a method for obtaining the information of gene expression, such as an EST analysis method, a SAGE method, and a DNA array method. Hence, the gene expression level in a mRNA sample used herein can be found. A list is prepared by numbering genes based on their expression levels and then highly expressed gene candidates to be eliminated are selected. A plurality (e.g., 2 or more types) of target genes are applied in most cases. For example, based on information based on the above database, 2 to 10, 2 to 50, 2 to 100, 2 to several hundred, 2 to 1000, 2 to several thousand, or an even higher number of target genes are selected, in descending order of expression levels.

A cDNA library having a reduced content of a highly expressed gene can be produced by the method of the present invention. Specifically, a cDNA library having an improved content of a gene, number of copies of which is about 12,000 per cell or less, or is preferably about 300 per cell or less, can be produced.

A probe that binds to the mRNA of a target gene has a property of blocking the extension of 1st strand cDNA to be synthesized by reverse transcriptase in the 5′ end direction on the mRNA strand.

The probe has the following characteristics.

### (1) Basic Structure

The basic structure of the probe is an oligonucleotide having a sequence complementary to a partial sequence of the mRNA sequence of a target gene, wherein the 3′ end is modified so that it does not serve as an initiation point for an extension reaction with reverse transcriptase. For example, the probe has a structure of dideoxyribonucleic acid (ddNTP) lacking a hydroxyl group or has a structure in which a hydroxyl group of a nucleotide at the 3′ end is bound to a compound such as biotin.

### (2) Binding Position

The nucleotide sequence of mRNA of a target gene to which a probe complementarily binds is designed based on nucleotide sequence information according to actual data or public database of subject gene sequences. At this time, a binding site for a probe is preferably located in a common region among variants since it is known that a plurality of transcript variants are generated from a single gene locus due to differences in promoter, splicing, poly (A) addition site, and the like.

### (3) Nucleotide Length of Nucleotide Sequence of Complementary Strand

The nucleotide length of a nucleotide sequence of a complementary strand is not particularly limited, as long as it is sufficient for stable binding to the mRNA of a target gene with a length of 20 to 50 nucleotides, preferably of 20 to 40 nucleotides, and further preferably of 20 to 35 nucleotides.

### (4) Introduction of Non-Natural Nucleic Acid

The term “non-natural nucleic acid” refers to a nucleic acid having a structure lacking natural nucleic acids, but having nucleotides with structures that are absent in the nature. In the present invention, introduction of one or a plurality of nucleotides that are absent in natural nucleic acids into a probe refers to introduction of a non-natural nucleic acid(s). The probe of the present invention is an oligonucleotide containing non-natural nucleic acids.

When non-natural nucleic acids are introduced into a probe, Tm value for the probe is preferably determined to be higher by 2° C. or more than the Tm value for a nucleotide sequence that is complementary to a partial sequence of the target sequence and is composed of natural nucleic acids. Also, even higher Tm value is more preferable. Also, Tm value for a probe must be higher than the temperature at which 1st strand cDNA is synthesized in step (ii). The Tm value for a nucleotide sequence composed of natural nucleic acids can be calculated by a known method. The probe is designed in such a manner so that after the probe has once annealed to the mRNA of a target gene, the probe is not easily separated, resulting in a greater effect of suppressing the extension reaction. Any nucleic acid may be used as a non-natural nucleic acid, as long as it complementarily binds to mRNA. Polyamide nucleic acids or locked nucleic acids, bridged nucleic acids (BNAs), or the like are preferably used, for example. Here, the term “polyamide nucleic acid” refers to a DNA analog containing neutral-amide main-chain bonding (e.g., Nielsen et al., Science 254: 1497, 1991). The term “locked nucleic acid” refers to a bicyclic nucleic acid analog in which an O-methylene (oxy-LNA), an S-methylene (thio-LNA), or an NH₂-methylene component (amino-LNA) binds to a furanose ring at positions 2′ and 4′. The term “bridged nucleic acid (BNA)” refers to an artificial synthetic nucleic acid prepared via cross-linking of natural nucleic acid molecules (e.g., JP Patent Publication (Kokai) No. 10-195098 A (1998)). When a locked nucleic acid is used, the Tm value resulting from the mixing of a natural type and a non-natural type can be predicted by referring to Niels Tolstrup's report (Niels Tolstrup et al., Nucleic Acid Research, 2003, 31, 3758-3762), for example. When a non-natural nucleic acid is introduced, preferably 2 or more nucleotides and further preferably 3 or more nucleotides from the 5′ end of the probe are non-natural nucleic acids. Also, non-natural nucleic acids to be introduced are preferably continuous. The upper limit of the number of non-natural nucleic acids to be introduced is not limited, but is preferably 10 or less and further preferably 5 or less.

A probe composed of such natural and non-natural nucleic acids can be chemically synthesized by a conventional method. Such probe to be used herein with a general degree of purification can be used.

When a plurality of target genes are present, a plurality of probes are used so that they can correspond to all of these target genes. The total number of probes to be used herein is at least the same as that of target gene types. A plurality of probes differing in positions of partial sequences may be used for one target gene.

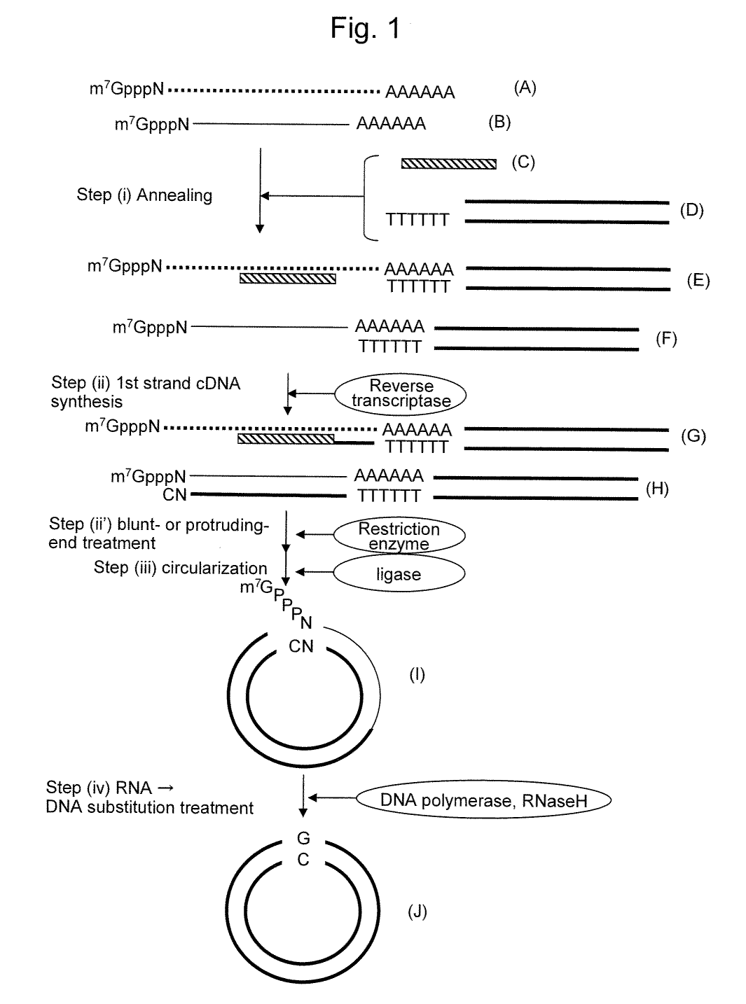

Steps of the present invention will be explained according to FIG. 1.

In step (i), a probe (C) binding to target gene mRNA (A) and a double-stranded DNA primer (D) having oligo dT is annealed to a sample RNA containing the target gene mRNA (A) and non-target gene mRNA (B). Therefore, a target gene mRNA (E) to which the probe (C) and the double-stranded DNA primer (D) are bound is generated, and a non-target gene mRNA (F) to which the double-stranded DNA primer (D) is bound is generated. cDNA can be synthesized with even less than 1 μg of such a sample RNA, but preferably 1 μg or more of a sample RNA is used. A single or 2 or more types of target gene can be used. Hence, a required number of types of the probe (C) having the above characteristics is prepared in accordance with the number of types of target genes. The amount of the probe (C) to be used herein is preferably 50-100 times (in terms of molar ratio) the amount of mRNA (A) or (B), but is not particularly limited thereto. Also, the number of consecutive dTs composing the sequence of the above double-stranded DNA primer (D) preferably ranges from 30 to 70. The double-stranded DNA primer (D) may be such primer containing a replication origin, or a replication origin and a cDNA expression promoter, but the examples thereof are not particularly limited thereto. A restriction enzyme site may be introduced in advance into the double-stranded DNA primer in accordance with the intended use of the cDNA. Annealing temperature may be determined based on the Tm value of the probe (C), so that it is lower than the Tm value of the probe (C).

Probe design and annealing conditions can be adequately determined depending on the types of target gene.

In step (ii), reverse transcriptase is caused to act on the target gene mRNA (E) to which the probe (C) and the double-stranded DNA primer (D) are bound, and the non-target gene mRNA (F) to which the double-stranded DNA primer (D) is bound, so that 1st strand cDNA complementary to the mRNA in the direction from the 3′ end to the 5′ end is synthesized. Preferable reverse transcriptase lacks endogenous RNaseH activity. In the case of the target gene mRNA (E) to which the probe (C) and the double-stranded DNA primer (D) are bound, 1st strand cDNA extension stops before the probe, resulting in an incomplete mRNA/DNA heteroduplex (G) that does not reach the 5′ end of the mRNA. Meanwhile, in the case of the non-target gene mRNA (F) to which the double-stranded DNA primer (D) is bound, a mRNA/DNA heteroduplex (H) is formed in which a complementary strand DNA is completely synthesized so as to reach the 5′ end. Therefore, 1st strand cDNA is synthesized from only the non-target gene mRNA. In step (ii), a mRNA/cDNA heteroduplex-to-double-stranded DNA primer conjugate is formed. In the conjugate, one end of a cDNA strand of the mRNA/cDNA heteroduplex and one end of a single strand of the double-stranded DNA primer are linked. Also, the cDNA of the mRNA/cDNA heteroduplex generated in step (ii) is complementary to mRNA having a cap structure and is a cap-consecutive cDNA containing dC or 5′-dC(dA)n-3′ added to the 3′ end or a mRNA-derived cap non-consecutive cDNA having no cap structure.

In step (ii′), other end of the double-stranded DNA primer ligated to the mRNA/DNA heteroduplex is blunt-ended or is preferably treated to have the 5′ protruding end through cleavage with a restriction enzyme. In this case, it is required to cause the terminal region of a double-stranded DNA primer to have a restriction enzyme site in advance. In addition, this step can be omitted. Implementation of this step can lead to a significant decrease in the background composed only of a vector contained in a cDNA library.

In step (iii), the mRNA/DNA heteroduplex-to-double-stranded DNA primer conjugate wherein other end of the double-stranded DNA primer has been blunt-ended or treated to have a 5′ protruding end is circularized with ligase. As ligase, various types of DNA ligase or RNA ligase can be used and T4RNA ligase is preferably used. A complete non-target gene mRNA/DNA heteroduplex (H), in which the 1st strand cDNA extends to the 5′ end of mRNA, is circularized with ligase. However, an incomplete target gene mRNA/DNA heteroduplex (G), in which 1st strand cDNA extension is not completed, is not circularized.

In step (iv), in the thus circularized non-target gene mRNA/DNA heteroduplex (H), the mRNA strand is degraded with RNaseH and the RNA strand is further substituted with a DNA strand with DNA polymerase and DNA ligase. Accordingly, non-target gene cDNA (J) is synthesized.

If the double-stranded DNA primer (D) contains a replication origin and a drug resistance gene, step (v) is carried out so that transformation into Escherichia coli or the like is performed and then the RNA strand may be substituted with a DNA strand in an in vivo reaction.

The thus obtained non-target gene double-stranded cDNA may be cloned into a vector. For example, after cleavage at a restriction enzyme site provided in a double-stranded DNA portion, the resultant may be inserted into a plasmid vector, a phage vector, or the like. At this time, through the use of a vector primer as a double-stranded DNA primer, a step of insertion into another vector can be omitted. A vector primer can be prepared by restrictively cleaving a circular vector DNA at an appropriate cloning site and then ligating a primer sequence complementary to a partial sequence of mRNA to the 3′ end, so as to form a 3′ protruding end.

A cDNA library obtained by the above method for constructing a cDNA library has a reduced content of target gene-derived cDNA clones. The content of individual target gene-derived cDNA clones accounts for 2% or less and preferably 1% or less of the total number of cDNA clones. Also, the percentage by which the number of target gene-derived clones is reduced when a probe has been added to the target gene is 60% or more, preferably 70% or more, and further preferably 80% or more. When a target gene is a highly expressed gene, the above cDNA library mainly contains lowly expressed gene-derived cDNAs. In addition, the cDNA library includes clones containing cap-consecutive cDNA with an extremely high probability of 60% or more, preferably 75% or more, further preferably 90% or more, and most preferably 95% or more.

A kit for constructing a cDNA library comprises the probe of the present invention to be annealed to a target gene and reagents for synthesis of other cDNAs. Examples of reagents for cDNA synthesis include a double-stranded DNA primer, reverse transcriptase, and T4 RNA ligase.

### EXAMPLES

Next, the invention will be described more specifically by referring to examples. The present invention is not limited to the examples. In addition, basic procedures concerning DNA recombination and enzyme reactions were carried out according to the literature (Sambrook and Maniatis, in Molecular Cloning—A Laboratory Manual, Cold spring Harbor Laboratory Press, New York, 1989). Restriction enzymes and various modification enzymes used herein were those produced by Takara Shuzo Co., Ltd., unless otherwise specified. Buffer compositions and reaction conditions for each enzyme reaction were employed according to relevant instructions.

### Example 1

### Suppression of cDNA Extension Reaction Via Probe Binding

### (1) Determination of Target Gene

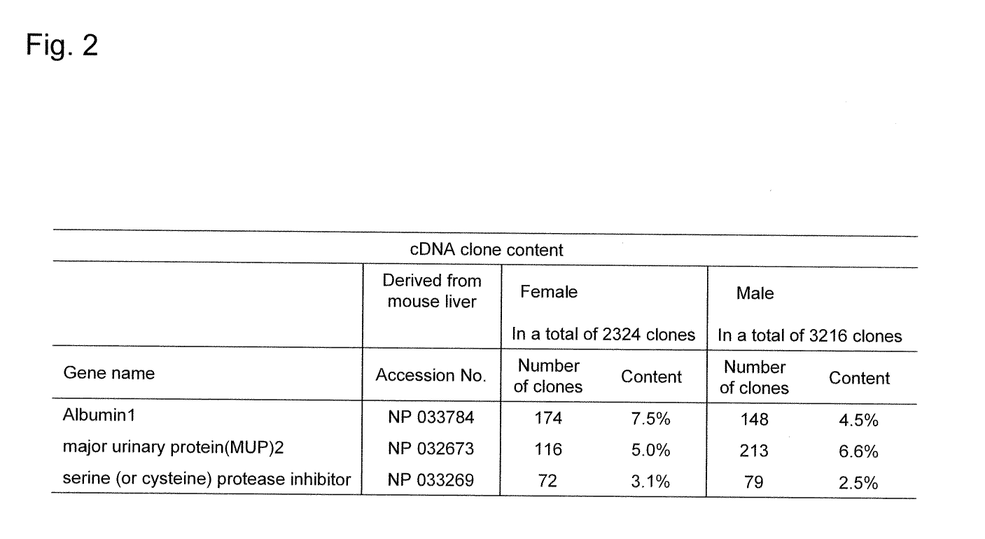

The following experiment was conducted to determine target genes with which experimental effects can be easily confirmed because of their high expression levels. cDNA synthesis was carried out using total RNA (10 μg each) obtained from a male or female mouse liver and 300 ng of pGCAP10 vector primer (International Patent Publication WO2004/087916 (Pamphlet)), so that cDNA clones were obtained. The preparation method was carried out according to description in International Patent Publication WO2004/087916 (Pamphlet). The 5′-end nucleotide sequences of the thus obtained cDNA clones were analyzed. The thus sequenced 3,217 clones derived from a male mouse liver and the thus sequenced 2,325 clones derived from a female mouse liver were subjected to BLAST search using a GenBank nucleic acid database. As a result, a Major Urinary Protein 2 (MUP2) gene was observed to exhibit the highest degree of overlapping (213 clones (6.6%)) among the cDNA clones obtained from a male mouse liver and the Albumin1 (Alb1) gene was observed to exhibit the highest degree of overlapping (174 clones (7.5%)) among the cDNA clones obtained from a female mouse liver (FIG. 2). Based on these results, the Alb1 gene and the MUP 2 gene were determined as target genes.

(2) mRNA Preparation

T7 RNA polymerase promoter is located upstream of the cDNA cloning site of the pGCAP10 vector and a Not I site is located downstream of the same. Hence, the thus obtained cDNA clones of the Alb1 gene and the MUP2 gene were linearized via Not I digestion. With the use of the resultants as templates and T7RNA polymerase, mRNA was prepared using an in vitro transcription kit (Ambion).

### (3) Probe Design

Probes for stopping a reverse transcription reaction, which is cDNA synthesis reaction, were designed.

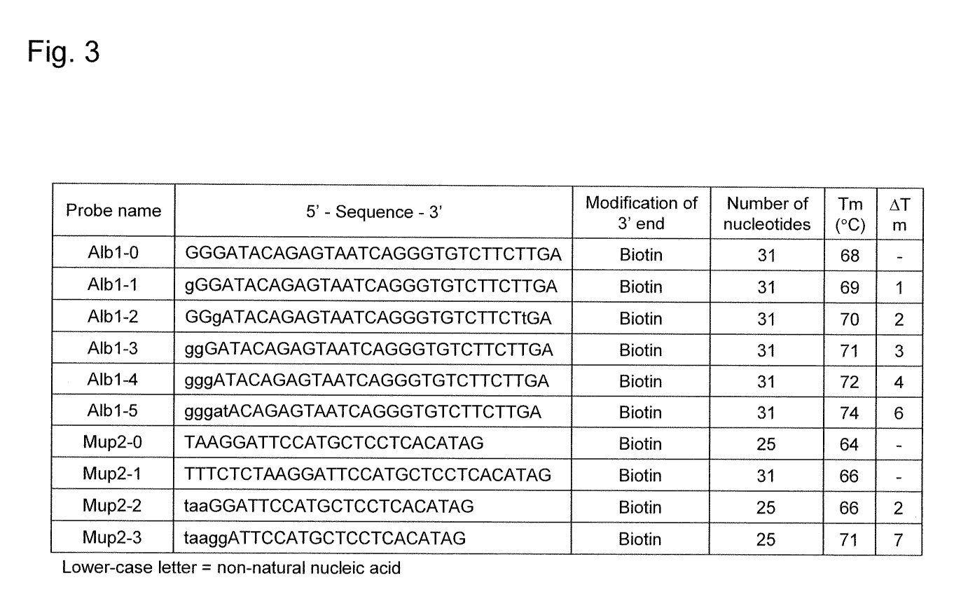

The sequences of translation regions of target gene mRNAs were employed as sequences of the probes. Also, as a non-natural nucleic acid, bridged nucleic acid (BNA) was arranged in the sequence of each probe. There are no examples or detailed information concerning the use of special nucleic acids such as BNA or PNA for stopping reverse transcription or other extension reactions. Examination was carried out by varying the number of BNAs, ranging from 1 to 5. Also, the 3′ end was modified with biotin, so as to prevent the probe from functioning as a primer for an extension reaction. The thus designed probe sequences are as shown in FIG. 3. In the sequences in FIG. 3, lower-case letters indicate bridged nucleic acids. The sequences represented by Alb1-0 and Mup-0 are natural nucleic acids. SEQ ID NOS: 1, 2, and 3 in the Sequence Listing represent the sequences of Alb1-0, Mup2-0, and Mup2-1, respectively. Also, we consigned probe synthesis to Gene Design Inc.

### (4) Verification of Effect of Suppressing Reaction

The effect of suppressing the reverse transcription reaction was examined using the probes prepared in (3) above. Probes having “Alb1” in their names were added when Alb1 mRNA was used as a template and probes having “MUP2” in their names were added when MUP2 mRNA was used as a template. cDNA synthesis was carried out for each case. As each reaction solution, a total of 13 μl of a reaction solution was prepared by mixing mRNA (500 ng), poly dT20 primer (100 pmol), dNTP (10 nmol), and a probe (100 pmol) of (3) above. A control reaction solution containing no probe of (3) above was prepared. These reaction solutions were each maintained at 65° C. for 5 minutes and then rapidly cooled on ice for 2 minutes. SuperScript II RNase H-reverse transcriptase (Invitrogen Corporation) (200 U), an attached reaction buffer (4 μl), DTT (100 nmol), and ribonuclease inhibitor (40 U) were added so that the volume of each reaction solution was 20 μl. After 1 hour of reaction at 42° C., 1st strand cDNA was synthesized. The reaction solutions were subjected to phenol extraction and then an mRNA/cDNA heteroduplex was collected by ethanol precipitation. The resultant was dissolved in water (10 μl) containing RNase A to degrade mRNA and then the length of cDNA strand in each reaction solution was confirmed by agarose gel electrophoresis. As a result, compared with the control, no stopping of reverse transcription reaction was observed for the Alb1-1 probe, Alb1-2 probe, or Mup 2-1 probe. However, cDNA synthesis was decreased by about 88% in the case of the Alb1-5 probe, by about 66% in the case of the Alb1-4 probe, by about 25% in the case of the Alb1-3 probe, and by about 50% in the cases of the Mup 2-3 probe and the Mup2-2 probe. Regarding conditions of the arrangement and number of BNAs contained in each probe required for suppression of a reverse transcription reaction, it was suggested by the experimental results that 3 or more consecutive nucleotides are effective, in the case of GC-rich sequences. Also, the effect of the MUP2 probe containing AT-rich BNA is weaker than that of the Alb1 probe. Hence, it was considered that conditions under which similar effects could be obtained with any sequence should be examined.

### (5) Optimization of Effect of Suppressing Reaction

Conditions for suppression of cDNA synthesis by Alb1 mRNA were examined using the Alb1-5 probe. Examination was carried out with regard to the amounts of probes to be used, annealing temperature, and annealing time. Reaction was carried out in the above mentioned reaction system by varying only the amount of the probe to be used. Also, regarding annealing temperature, an attached reaction buffer (4 μl), DTT (100 nmol), and ribonuclease inhibitor (40 U) were added so that the volume of the reaction solution was 19 and then the reaction solution was maintained at 42° C. or 57° C. for 30 minutes to 2 hours. After rapid cooling, SuperScript II RNase H-reverse transcriptase (Invitrogen Corporation) (200 U) was added and then reaction was carried out at 42° C. for 1 hour, so that cDNA was synthesized. The detection method was carried out in a manner similar to that in (4) above. As a result, the effect of suppressing reaction by 95% or more was the highest and exhibited under the following conditions: the amount of the probe used was 200 pmol, the annealing temperature was 57° C., and the annealing time was 2 hours. However, the following 3 conditions were determined as optimum conditions under which the effect of suppressing reaction by 90% or more could be obtained in view of the stability of RNA and the amounts of unbound probes, for example: (i) the amount of a probe to be used ranges from 50 to 100 pmol, (ii) the annealing temperature is 57° C., and (iii) the annealing time is 30 minutes.

Next, the effect of suppressing reaction was examined under the above optimum conditions using the Mup2-3 probe or the Mup 2-2 probe with which an effect equivalent to that in the case of the Alb1-5 probe had not been obtained. The effect of suppressing reaction by 90% or more was exerted in the case of the Mup2-3 probe. However, in the case of the Mup2-2 probe, the effect of suppressing reaction by about 50% was exerted, which was similar to that in the previous case. These results suggested that regarding the effect of suppressing reaction under the optimum conditions employed in this experiment, suppression by 90% or more was observed regardless of sequence as long as a probe containing 5 consecutive BNAs had been used. Also, it was suggested that: a probe that is preferably used for the effect of suppressing reaction has a Tm value (ΔTm) higher by 2° C. (or more) than that of a probe prepared using natural nucleic acids and contains 2 or more consecutive non-natural nucleic acids from the 5′ end; and the higher the ΔTm, the higher the effect of suppressing reaction.

### Example 2

### Construction of cDNA Library Having Reduced Content of Highly Expressed Gene

(1) 1st Strand cDNA Synthesis

cDNA synthesis was carried out from total RNA using the above probes. The probes used herein were Alb1-5 and Mup2-3. Total RNA (10 μg each) obtained from the liver of a male mouse, a pGCAP10 vector primer (JP Patent Publication (Kokai) No. 4-108385 A (1992)) (150 ng), dNTP (25 nmol), and 100 pmol probe were mixed, so that a total of 8 μl of each mixture was prepared. The mixed solution was heated at 65° C. for 5 minutes and then ice-cooled for 2 minutes. An attached buffer (4 μl) of SuperScript II RNase H-reverse transcriptase (Invitrogen Corporation) was added and then the solution was maintained at 57° C. for 30 minutes. Subsequently, the solution was ice-cooled for 2 minutes, mixed with DTT (100 nmol), ribonuclease inhibitor (40 U), and SuperScript II RNase H-reverse transcriptase (Invitrogen Corporation) (200 U), so as to adjust the solution to a total volume of 20 μl. The mixed solution was subjected to 1 hour of reaction at 42° C., so that 1st strand cDNA was synthesized. After phenol extraction for the reaction solution, the conjugate of mRNA/cDNA heteroduplex and the vector primer was collected by ethanol precipitation and then dissolved in 80 μl of water.

### (2) Self Ligation

10×H buffer (TAKARA SHUZO Co., Ltd.) and 50 U of restriction enzyme EcoR I (TAKARA SHUZO Co., Ltd.) were added to 80 μl of the solution of the conjugate of a mRNA/cDNA heteroduplex and a vector primer to a total volume of 100 μl, followed by 1 hour of reaction at 37° C. The solution was subjected to phenol extraction, the conjugate of the mRNA/cDNA heteroduplex and the vector primer treated with a restriction enzyme was collected by ethanol precipitation, and then the resultant was dissolved in 24 μl of water. The solution was mixed with a reaction solution (50 mM Tris-HC 1 (pH 7.5), 5 mM MgCl₂, 10 mM 2-mercaptoethanol, 0.5 mM ATP, and 2 mM DTT), 120 U of T4 RNA ligase (TAKARA SHUZO Co., Ltd.) was added, followed by 16 hours of reaction at 20° C. The mRNA/cDNA heteroduplex was ligated to EcoR I end of the vector primer for circularization (self ligation reaction). The reaction solution was subjected to phenol extraction, a self ligation product was collected by ethanol precipitation, and then the self ligation product was dissolved in 50 μl of water, so that a cDNA vector solution was prepared.

(3) Transformation of Escherichia coli

The thus prepared cDNA vector solution (1 μl) was mixed with 20 μl of DH10B Electro-cells (Invitrogen Corporation), so that transformation was carried out by an electroporation method. Electroporation was carried out using MicroPulser (Bio-Rad Laboratories Inc.). The thus obtained transformant was suspended in SOC medium, seeded on LB agar medium containing 50 μg/ml ampicillin, and then cultured at 35° C. for 16 to 18 hours, so that cDNA clone-transformed Escherichia coli was obtained.

### (4) Calculation of the Content by Hybridization

The following 4 types of library were constructed.

C: Library (C) to which no probe was added.
Alb: Library (Alb) to which Alb1-5 probe was added.
MUP: Library (MUP) to which Mup2-3 probe was added.
W: Library (W) to which Alb1-5 and Mup2-3 probes were added

To find the content of the Alb1 gene (GenBank Accession No. NP_{—}033784) and the content of the MUP 2 gene (GenBank Accession No. NP_{—}032673) contained in the constructed cDNA libraries, about 4000 clones were sampled from each library and then subjected to colony hybridization.

DIG DNA-labeled probe for hybridization was prepared as follows. The Alb1 gene was digested with Bst X I and Not I restriction enzymes, so that a 1.1-kbp fragment was generated. Also the MUP2 gene was digested with Avr II and Ava II restriction enzymes, so that a 0.7-kbp fragment was generated. These fragments were subjected to agarose gel electrophoresis, excised, and then increased in amounts by PCR. Subsequently, the DIG DNA-labeled probe was prepared using a DIG High Prime DNA Labeling and Detection Starter Kit I (Roche Applied Science). Next, about 4000 colonies were transferred from each library to a Hybond N+nylon membrane (Amersham). After alkali fixation, hybridization was carried out. Hybridization and detection were carried out according to protocols using DIG High Prime DNA Labeling and Detection Starter Kit I (Roche Applied Science). Hybridization was carried out at 42° C.

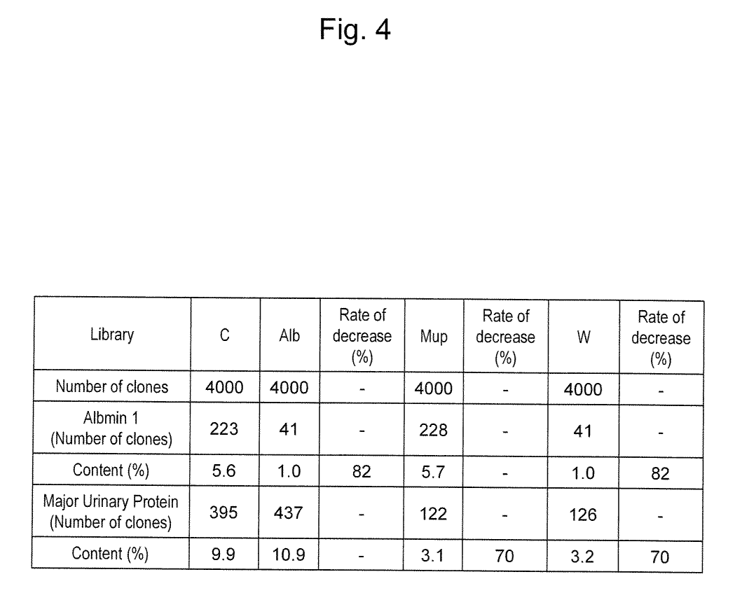

The number and content of clones detected are shown in FIG. 4. The content of Alb1 gene clones decreased from 5.6% to 1.0% in the case of Alb library and also decreased to 1.0% in the case of W library. Therefore, the rate of decrease was 82% in all cases. Also, the content of MUP2 gene clones decreased from 9.9% to 3.1% in the case of MUP library and decreased to 3.2% in the case of W library. Therefore, the rate of decrease was about 70% in all cases. These results suggest that a decrease by 70% or more can be expected with the addition of the probe. Also, in the case of the W library, the reduction by the same percentage was obtained for each gene even when 2 types of probe had been added.

### (5) Calculation of Content Via Analysis of 5′-End Nucleotide Sequence

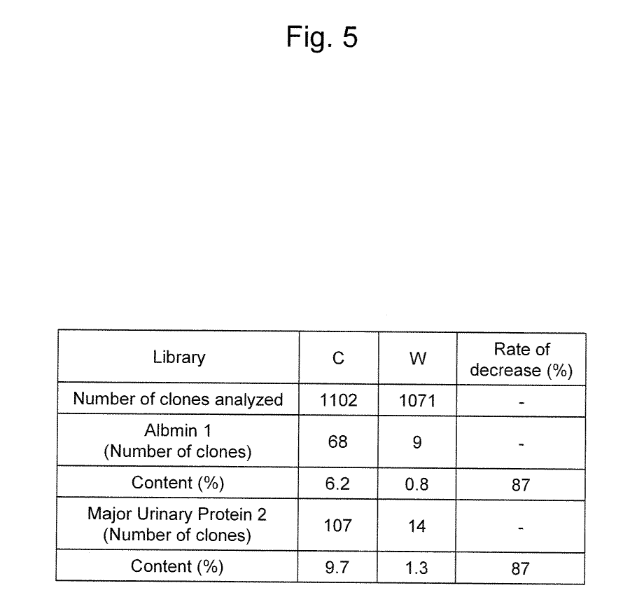

The structural gene region of the nucleotide sequence of the Mup2 gene has 80% or more homology with that of the other Mup genes (Mup1 and Mup3). Therefore, not only the Mup 2 gene, but also the entire Mup group was detected by hybridization. Hence, to find the content more precisely, the nucleotide sequences of about 1000 clones in C and W libraries were analyzed and then numbers of clones, contents, and rates of decreases were found (FIG. 5). As a result, the rate of decrease for Alb1 or for Mup2 was found to be 87%.

As a result of calculation of contents by hybridization and nucleotide sequence analysis above, it was demonstrated that addition of a probe(s) satisfying requirements that should be satisfied by the probes of the present invention and construction of a cDNA library using a double-stranded DNA primer decrease the content of clones corresponding to the probe(s) by 80% or more and such decreasing effect can be obtained by addition of a plurality of probes.

All publications, patents, and patent applications cited in this description are herein incorporated by reference in their entirety.

### SEQUENCE LISTING FREE TEXT

SEQ ID NOS: 1-3 Synthesis

1. A method for constructing a cDNA library having a reduced content of cDNA clones derived from a target gene, comprising the steps of:
(i) annealing a double-stranded DNA primer to an RNA mixture containing mRNA having a cap structure at the 5′ end and then further annealing at least one probe that binds to mRNA of the target gene so as to inhibit the reaction of reverse transcriptase;
(ii) synthesizing 1st strand cDNA from the double-stranded DNA primer using reverse transcriptase and thus preparing a conjugate of a mRNA/cDNA heteroduplex and the double-stranded DNA primer; and
(iii) ligating the 3′ end to the 5′ end of the DNA strand containing the cDNA of the conjugate of the mRNA/cDNA heteroduplex and the double-stranded DNA primer using ligase for circularization.

2. The method according to claim 1, wherein the target gene is a highly expressed gene.

3. The method according to claim 1, comprising a step of selecting the target gene based on a gene database.

4. The method according to claim 1, wherein the mRNA having the cap structure is contained in a cell extract.

5. The method according to claim 1, wherein the primer sequence of a double-stranded DNA primer contains a sequence complementary to the poly (A) sequence of mRNA having a cap structure.

6. The method according to claim 1, wherein the ligase is T4RNA ligase.

7. The method according to claim 1, further comprising, between step (ii) and step (iii), step (ii′) of cleaving the conjugate of the mRNA/cDNA heteroduplex and the double-stranded DNA primer with a restriction enzyme, so as to generate a 5′ protruding end or blunt end of the double-stranded DNA primer.

8. The method according to claim 1, further comprising, following step (iii), step (iv) of substituting the RNA strand of the conjugate of the mRNA/cDNA heteroduplex and the double-stranded DNA primer with a DNA strand.

9. The method according to claim 1, wherein the double-stranded DNA primer contains a replication origin, or a replication origin and a cDNA expression promoter.

10. The method according to claim 1, wherein the probe that binds to the mRNA of a target gene so as to inhibit a reaction with reverse transcriptase is an oligonucleotide containing non-natural nucleic acids with a melting temperature (Tm value) higher by 2° C. or more than that of a complementary sequence of a partial sequence of the target gene.

11. The method according to claim 1, wherein the probe that binds to the mRNA of a target gene so as to inhibit a reaction with reverse transcriptase is an oligonucleotide in which 2 or more consecutive nucleotides from the 5′ end side are composed of non-natural nucleic acids.

12. The method according to claim 10, wherein the non-natural nucleic acids are locked nucleic acids, polyamide nucleic acids, or bridged nucleic acids.

13. The method according to claim 1, wherein the probes that bind to the mRNAs of target genes so as to inhibit a reaction with reverse transcriptase target the mRNAs of different types of target gene.

14. A probe having a sequence complementary to an mRNA sequence, which comprises an oligonucleotide that is designed so that the 3′ end has a structure modified so that it does not serve as the initiation point for an extension reaction with reverse transcriptase, 2 or more consecutive nucleotides from the 5′ end are non-natural nucleic acids, and the Tm value of the nucleotide sequence of the entire probe is higher by 2° C. or more than that of a nucleotide sequence composed of only natural nucleic acids.

15. The probe according to claim 14, which is used for constructing a cDNA library having a reduced content of cDNA clones derived from a target gene and binds to the mRNA of the target gene so as to inhibit a reaction with reverse transcriptase.

16. A reagent kit for producing a cDNA library containing the probe according to claim 14, a double-stranded DNA primer, reverse transcriptase, and T4 RNA ligase.


SEQUENCE LISTING (3 sequences)

SEQ ID NO: 1 - DNA, 31 bp, Artificial
  chemically synthesized
gggatacagagtaatcagggtgtcttcttga

SEQ ID NO: 2 - DNA, 25 bp, Artificial Sequence
  chemically synthesized
taaggattccatgctcctcacatag

SEQ ID NO: 3 - DNA, 31 bp, Artificial Sequence
  chemically synthesized
tttctctaaggattccatgctcctcacatag